(19) United States
(12) Patent Application Publication
(10) Pub. No.: US 2017/0296390 A1
(43) Pub. Date: Oct. 19, 2017

(54) LIMB JACKET WRAP DRESSING

(71) Applicants: Ivy Swanson Razmus, Tulsa, OK (US); Mary Rebecca Koterba, Tulsa, OK (US)

(72) Inventors: Ivy Swanson Razmus, Tulsa, OK (US); Mary Rebecca Koterba, Tulsa, OK (US)

(21) Appl. No.: 15/489,070

(22) Filed: Apr. 17, 2017

Related U.S. Application Data
(60) Provisional application No. 62/324,298, filed on Apr. 18, 2016.

(51) Int. Cl. A61F 13/00; A61M 1/00

(52) U.S. Cl. CPC A61F 13/00029; A61M 1/008; A61F 13/00068; A61F 13/00034; A61F 13/00063; A61F 2013/00217

(57) ABSTRACT

A wrap for treating wounds of a patient is disclosed. The wrap may include a first material layer and a second material layer. The wrap may also include a first pocket formed between the first material layer and the second material layer. A dressing may be located within the first pocket, between the first material layer and the second material layer and a first fluid port may extend between the first material layer to the first pocket.

### CROSS-REFERENCE

This application is a continuation application of Ser. No. 62/324,298, filed Apr. 18, 2016, which is incorporated herein by reference in its entirety and to which application we claim priority under 35 USC §120.

### BACKGROUND

Ulcers are wounds or open sores that will not heal or keep returning. Many of the patients have chronic wounds with a combination of factors leading to the progression of lower leg ulcers. Physicians presented with such patients decide between performing invasive procedures for limb salvage and avoiding the risk of aggressive unnecessary treatment on patients who may have a cardiovascular risk.

Invasive procedures for limb salvage include treatment options such as valvuloplasty, venous bypass, external banding devices, thermally induced collagen shrinking, ablation of refluxing veins, percutaneous transluminal angioplasty, intravascular stents, intra-arterial thrombolysis, and arterial bypass surgery. The greatest risk for these patients however is a delay in treatment for infection or debridement.

Clinical symptoms associated with lower leg ulcers include leg pain, edema, skin changes such as hyperpigmentation. Lower leg ulcers can be caused by a variety of factors that include venous stasis, arterial insufficiency, neuropathic, lymphadermatosclerosis and/or a mixture of these factors.

The most common etiologic factors responsible for chronic delayed healing among lower extremity wound are chronic venous insufficiency (CVI), diabetic neuropathy, and arterial insufficiency (AI). One or more of these factors can be identified in more than 90% of chronic lower extremity ulcers. These patients present with open weeping ulcers in need of treatment. For those patients who are not candidates for invasive therapies there is a need for more research evidence to support less invasive treatment modalities in healing these ulcers.

Negative pressure wound therapy may be used to treat wounds on a patient. The treatment includes the use of a sealed or occluded cover that is placed over the wound and seals the wound off from the environment outside the cover and relies on expensive vacuum machines to control the pressure applied to the wound. Occluded negative pressure wound therapy may be helpful in the treatment and healing of some wounds, but in some cases is inadequate and fails to cure all wounds.

### SUMMARY

A wrap for treating wounds of a patient is disclosed. The wrap may include a first material layer and a second material layer. The wrap may also include a first pocket formed between the first material layer and the second material layer. A dressing may be located within the first pocket, between the first material layer and the second material layer and a first fluid port may extend between the first material layer to the first pocket.

In some embodiments, the first material layer is a non-permeable layer.

In some embodiments, the second material layer is a permeable layer.

In some embodiments, wherein the first material layer is a non-permeable layer and the second material layer is a permeable layer.

In some embodiments, fenestrations may be formed through the second layer, the fenestrations coupling the first pocket in fluid communication with an environment outside the pocket.

In some embodiments, fenestrations may be formed through the second layer.

In some embodiments, the dressing may include saline solution.

In some embodiments, a first tie may extend from a first end of the wrap and a second tie extending from a second end of the wrap, the ties may be configured to knot together to secure the wrap to a limb of a patient.

In some embodiments, the wrap may include a re-sealable closure configured to open to provide access to the dressing.

In some embodiments, the wrap may include a second pocket, separate from the first pocket formed between the first material layer and the second material layer. A second dressing may be located within the second pocket, between the first material layer and the second material layer, and a second fluid port may extend through the first material layer to the second pocket.

A system for treatment of wounds of a patient is also disclosed. The system may include, a vacuum source and a wrap. The wrap may include a first pocket in fluid communication with an environment external to the first pocket, a dressing located within the first pocket, and a first fluid port coupling the first pocket in fluid communication with the vacuum source. The wrap may include a fluid separator between the vacuum source and the wrap and coupled in fluid communication with the vacuum source and the wrap.

In some embodiments, the fluid separator is a filter.

In some embodiments, the fluid separator is a fluid trap.

In some embodiments, a permeable material defines a portion of a boundary of the first pocket and couples the first pocket in fluid communication with the environment external to the pocket.

In some embodiments, fenestrations in a material layer that defines a portion of a boundary of the first pocket couples the first pocket in fluid communication with an environment external to the first pocket.

In some embodiments, the system may include a second pocket, mechanically coupled to, but fluidically separated from the first pocket, a second dressing located within the second pocket, and a second fluid port coupling the second pocket in fluid communication with the vacuum source.

In some embodiments, a boundary defining the pocket is formed from a non-permeable.

In some embodiments, the system includes a re-sealable closure configured to open to provide access to the first pocket.

In some embodiments, the wrap includes a plurality of ties extending from first and second ends of the wrap.

In some embodiments, a first tube connects the wrap in fluid communication with the fluid separator and a second tube connecting the fluid separator with the vacuum source.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a top view of a limb wrap dressing, according to one or more embodiments disclosed herein.

FIG. 1B shows a bottom view of the limb wrap dressing of FIG. 1A, according to one or more embodiments disclosed herein.

FIG. 1C shows a side cross-section view about line C-C of the limb wrap dressing of FIG. 1B, according to one or more embodiments disclosed herein.

FIG. 2 shows a cross-section view of a limb wrapped with a limb wrap dressing, according to one or more embodiments disclosed herein.

FIG. 3 shows a system for treating wounds, according to one or more embodiments herein.

FIG. 4A shows a top view of a limb wrap dressing, according to one or more embodiments disclosed herein.

FIG. 4B shows a side cross-section view about line B-B of the limb wrap dressing of FIG. 2AB, according to one or more embodiments disclosed herein.

FIG. 5 shows a top view of a limb wrap dressing, according to one or more embodiments disclosed herein.

FIG. 6 shows a bottom view of the limb wrap dressing, according to one or more embodiments disclosed herein.

### DETAILED DESCRIPTION OF THE INVENTION

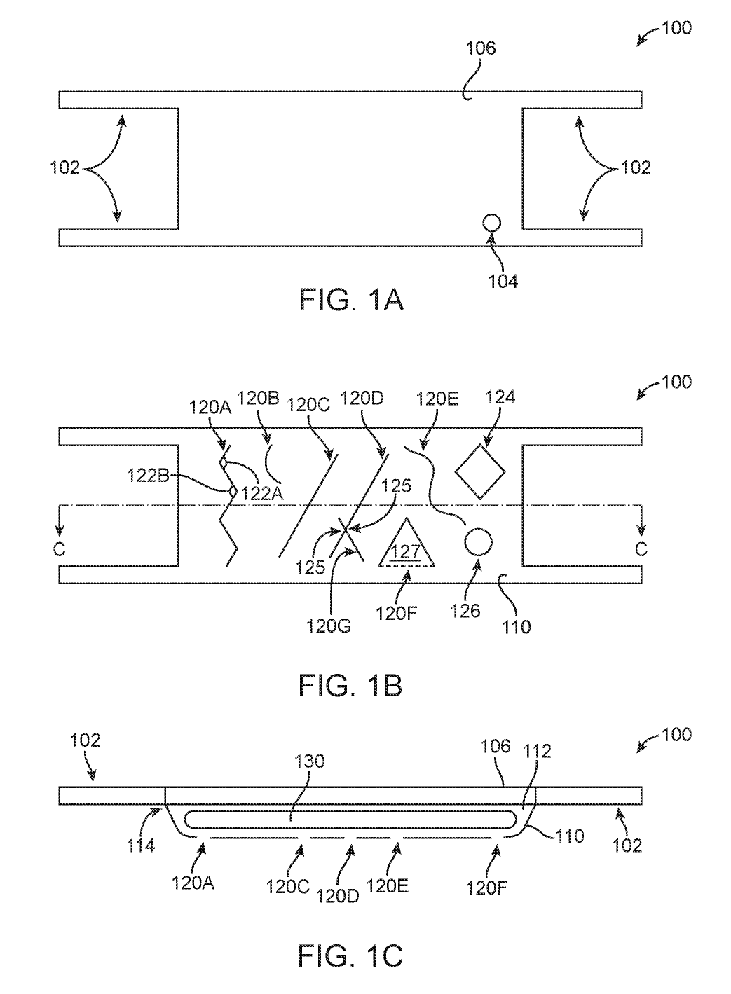

FIGS. 1A-1C show a jacket wrap dressing 100 according to one or more embodiments disclosed herein. As shown in FIGS. 1A-1C, the wrap 100 includes an upper material layer 106 coupled to a lower material layer 110. The upper material layer 106 may also be called the outward facing layer and the lower layer 110 may be called the patient facing layer because, during use, the upper material layer 106 faces outward or away from the patient while the lower layer 110 faces the patient's body and may be placed against the patient's body, for example, as described herein with respect to FIG. 5.

The upper material layer 106 may be a thin sheet of material. In some embodiments, the wrap 100 may be reusable, in such embodiments, the upper material layer 106 may be a thin polymer material, such as plastic or a non-permeable vinyl for reusable type wraps. In some embodiments, the wrap 100 may be disposable, in such embodiments, the upper material layer 106 may be a polymer, paper, non-woven, or woven layer. The upper material layer is preferably a non-permeable layer that resists or prevents the flow of fluids, such as air and liquids, from passing though.

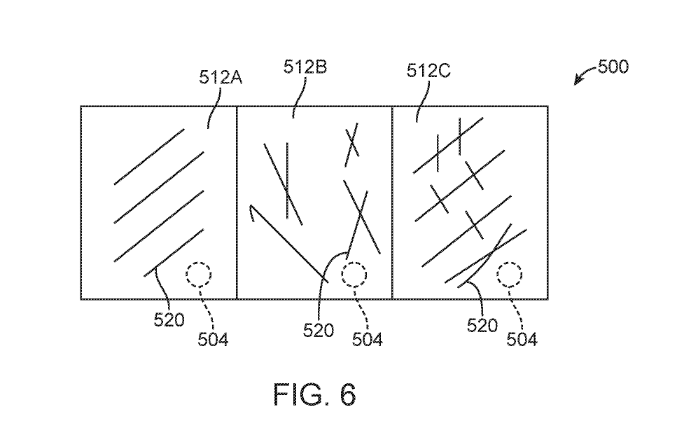

The upper layer may include a port, such as port 104. The port couples the inner pocket 112 of the wrap 100 in fluid communication with the environment outside the inner pocket 112 or with equipment which may be connected to the port 104. For example, as shown in FIG. 6, a vacuum source 630 may be coupled to the port 602 via tubing 610, such that a vacuum may be formed within the pocket 112.

The lower material layer 110 may also be a thin sheet of material. In some embodiments, the wrap 100 may be reusable, in such embodiments, the lower material layer 110 may be a thin polymer material, such as plastic or a non-permeable vinyl for reusable type wraps. In some embodiments, the wrap 100 may be disposable, in such embodiments, the lower material layer 110 may be a polymer, paper, non-woven, or woven layer. The lower material layer 110 may be a non-permeable layer that resists or prevents the flow of fluids, such as air and liquids, from passing though or a permeable layer of material that allows for the transfer of fluids, such as air and liquids.

The lower layer 110 may also be non-adhered, such that it resists adherence to skin or wounds of a patient. In some embodiments, the lower layer 110 may be formed from a non-adherent material while in some embodiments, the lower layer 110 may be coated with a non-adherent coating that, when applied to the lower layer 110, causes the lower later to resist adherence to skin or wounds of a patient.

In either reusable or disposable wraps 100 with permeable or non-permeable lower layers 110, the lower layer 110 may include fenestrations 120, which may be cuts, slits, or other types of openings though the lower material layer 110 that allow for passage of fluids through the lower material layer 110. The fenestration 120 may be of one or more types. For example, as shown in FIG. 1B, the fenestration may include a zig-zag shape wherein a plurality of sections of the fenestration 120A are connected at obtuse angles 122A, 122B with each other.

The wrap may also include curved or arc shaped fenestrations, such as fenestration 120B. These curved or arc fenestrations may have a radius of curvature of 0.1 mm, 0.25 mm, 0.5 mm, 0.75 mm, 1 mm, 1.5 mm, 2 mm, 3 mm, 5 mm, 7 mm, or 10 mm. In some embodiments, the radius of curvature may be between 1 mm and 10 mm. In some embodiments, the radius of curvature may be between 5 mm and 15 mm or between 10 mm and 30 mm. The arc of the curved or arch shaped fenestrations 120B may be between 10 degrees and 180 degrees. In some embodiments, the arc length may be less than 270 degrees, 180 degrees, 90 degrees, 60 degrees, 45 degrees, 30 degrees, 15 degrees or 5 degrees.

The wrap may also include linear fenestrations, such as fenestrations 120C, 120D and 120G. The linear fenestrations 120C, 120D and 120G may have a length of about 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm. In some embodiments, the linear fenestrations 120C, 120D and 120G may have a length of less than 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm.

In some embodiments, two fenestrations 120 may be parallel to each other. For example, the fenestrations 120C, 120D are parallel to each other. In some embodiments, fenestration 120 may intersect each other. For example, fenestrations 120D, 120G intersect each other. These intersecting fenestrations 120D, 120G create flaps 125 between the intersecting fenestrations 120D, 120G.

In some embodiments, the wrap 100 may also include wave shaped fenestrations, such as wave shaped fenestration 120E. The wave shaped fenestration 120E may be a series of arc shaped fenestrations connected to each other, or may take the shape of a sine wave, for example. In some embodiments, a wave fenestration may have an amplitude of about 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm. In some embodiments, a wave fenestration may have an amplitude of less than 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm. In some embodiments, a wave fenestration may have a wavelength of about 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm. In some embodiments, a wave fenestration may have a wave length of less than 0.1 mm, 0.25 mm, 0.5 mm, 0.75 mm, 1 mm, 1.5 mm, 2 mm, 3 mm, 5 mm, 7 mm, 10 mm 15 mm, 30 mm, 50 mm, 100 mm.

In some embodiments, a fenestration 120F may be formed from two intersecting cuts to form a triangular shaped flap 127.

In some embodiments, the lower material layer 110 may include apertures 124, 126. The apertures 124, 126 may be polygonal in shape, such as the quadrilateral shaped square aperture 124, or may be a curved shape, such as curved aperture 126, which is a circular, but may also be other shapes such as ellipses, ovals, etc.

The fenestrations 120 and apertures 124, 126 couple the interior pocket 112 in fluid communication with the environment outside the lower material layer. In some embodiments, the lower material layer 106 faces or is in contact with a wound.

The pocket 112, also called a cavity, is formed between the upper material layer 106 and the lower material layer 110. The outer portion 114, such as an outer perimeter of the lower material layer 110 may be coupled to an outer perimeter, such as an outer portion of the upper material later to form the pocket 112.

The pocket may include a dressing, such as the dressing 130. The dressing 130 may be made from an absorbent material such that it aids in retaining liquids removed from a wound in the pocket 112. The may be one or more of gauze, pregnated gauze, hydrofera blue, hydrofiber, calcium alginate, collagen, silicone foam. In some embodiments, the dressing may be a sponge dressings that may include one or more agents or medications for wound care treatment, such as silver, silvadene, santyl, normagel, normagel ag, regrenex, antibiotic ointment, bactroban, dakins solution, medihoney, normal saline, betadine, collagen, or tissue growth components.

In some embodiments, a saline solution may be applied to the dressing before or during use.

The wrap 100 may also include fasteners 102 that aid in securing the wrap 100 to a limb of a patient. The fasteners 102 shown in FIGS. 1A, 1B, and 1C are ties that can be knotted together to aid in securing the wrap to a patient.

The size, for example the length and width of the wrap can be different for different embodiments. The lengths and widths can be based on the size of the wound and the size of the extremity or area effected. The wrap, for example, may be as small as 1 cm×1 cm or smaller if it is to be used around a toe or finger and as large as 270 cm×270 cm plus if it is to be used around a large extremity or an abdomen of a patient. The size for use may be dependent upon the size of the patient's size and wound size. The dressing 112 may encompass the entire length of the wrap pocket 112 or the dressing 112 may be less than the size of the wrap pocket 112.

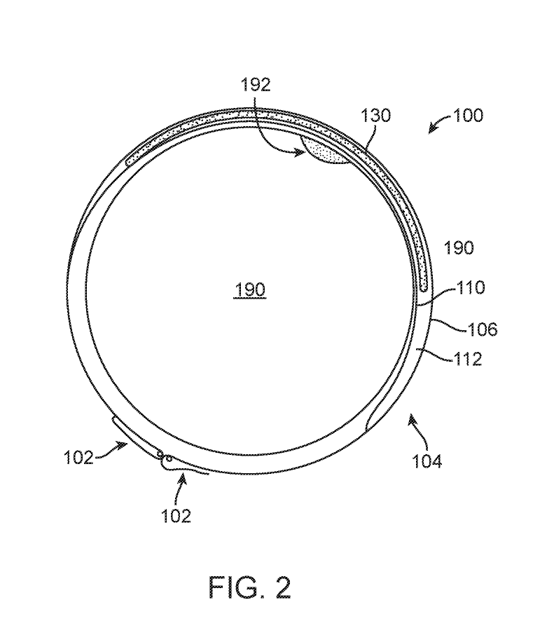

FIG. 2 shows the wrap 100 wrapped around and secured to a limb 190 of a patient. The limb 109 includes an external wound 192. In use, the wrap 100 is positioned over the wound 192 such that the pocket 112 and dressing 130 are placed over or directly over the wound 192 and then the wrap 100 is secured to the limb, for example by knotting or otherwise securing the ties 102 together.

With the wrap 100 secured to the patient at a wound location, a suction or vacuum may be applied to the pocket 112 via the port 104. The suction or vacuum is then applied to the wound via the a permeable lower material layer 110 and/or striations in the permeable lower material layer.

The wrap 100 may not seal around the wound. Unlike negative pressure wound therapy, where a seal is formed around the wound, in the treatment described herein, ambient air, or air from the environment outside the wrap 100 may flow between the wrap 100 and the patient, into the pocket 112, and then out the port 104.

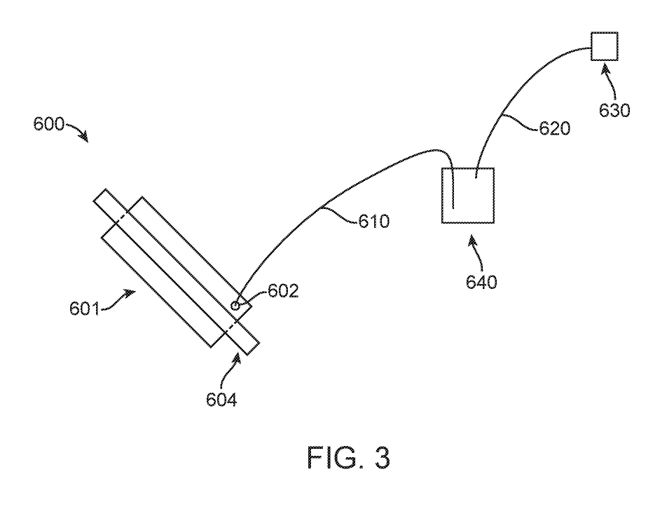

FIG. 3 shows an embodiment of a system 600 for use with a wrap 601, which may be similar to the wrap 100 or the other wraps discussed herein. The wrap 601 may be secured to a patient. For example, an elastic bandage, such as an ACE bandage, may be wrapped around the wrap 601 and the limb of the patient to secure the wrap 601 to the patient.

The wrap 601, and more particularly, the pocket within the wrap 601 is coupled in fluid communication with a vacuum source 630. In particular, a port 602 of the wrap 601 is coupled to a vacuum tube 610. The vacuum tube 610 may be coupled to a fluid separator 640. The fluid separator 640 separates or otherwise removes fluid from the vacuum tube 610 and aids in preventing fluid from reaching the vacuum source 630. The fluid separator 640 may be a filter, such as in inline filter that captures and retains fluid form the tube 610. In some embodiments, the fluid separator 640 may be a fluid trap.

A second vacuum tube 620 couples the vacuum tube 610 in fluid communication with the vacuum source 630. The vacuum source may be in-wall vacuum provided via a hospital's distributed vacuum system or an individual vacuum, such as a portable or in room vacuum.

During operation, the vacuum source 630 creates a partial vacuum. In some embodiments, the partial vacuum at the vacuum source 630 is between approximately 100 mmHg and 140 mmHg while the partial vacuum at the wound is between approximately 25 mmHg and 35 mmHg and more preferably about 30 mmHg, for example, between 29 mmHg and 31 mmHg. In some embodiments, the partial vacuum at the vacuum source may be in a range between 25 mmHg and 100 mmHg.

The vacuum source 630 pulls in air from the environment surrounding the wrap 601, past the wound and then through the lower material layer of the wrap 601. The air may be pulled through fenestrations, such as fenestrations 120, or other apertures, such as apertures 124, 126 in the lower material layer, such as material layer 110 of the wrap 601. In some embodiments, the lower material layer may be a fluid permeable layer through which the air flows. The air and entrained body fluids from the wound may than pass though the dressing, for example dressing 130, in the pocket, for example pocket 112, of the wrap 601. The air and entrained fluid may than pass through a port, such as port 104 and then through one or more vacuum tubes and fluid separators, before reaching the vacuum source 630.

Although depicted as a having a single vacuum source 630 connected to a single fluid separator and a single warp 601, in some embodiments, a single vacuum source 630 may be connected to one or more fluid separators which may each be connected to one or more wraps.

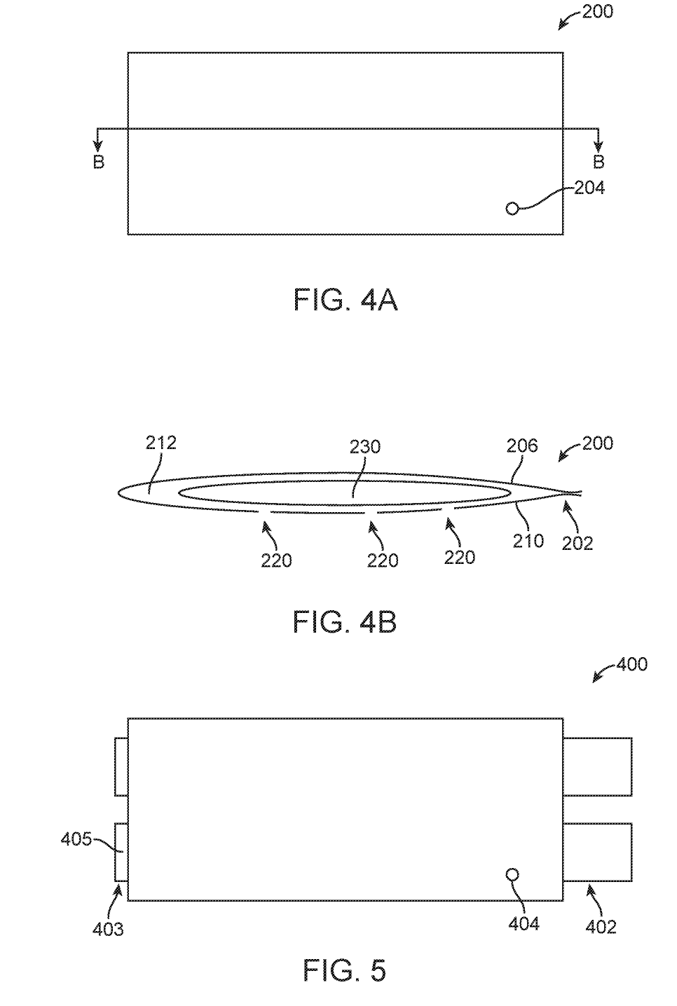

FIGS. 4A and 4B show embodiments of a wrap 200 without ties. The wrap 200 may be of a simple design, for example, that of a sealable bag. The upper and lower material layers 206, 210 may be sidewalls of the bag, for example a plastic bag. The upper and lower material layers 206, 210 may be permanently sealed or coupled at three edges with a single, fourth edge including a reusable, re-sealable closure 202. During use, the re-sealable closure 202 may be opened for the insertion and removal of the dressing 230.

Similar to the wrap 100, the wrap 200 includes a port 204 that is fluidly couplable to a vacuums source. The wrap 100 includes an inner pocket 112 that may include a dressing 230 therein. The lower material layer 210 may include fenestrations 220 or apertures, similar to those discussed above with respect to FIGS. 1A, 1B, and 1C.

FIG. 5 shows an embodiment of a wrap 400 with a hook and loop type closure. The straps 402 include at least a portion of a hook and loop coupling. In some embodiments, a first side the straps 402 include hooks and the second side include loops. When the wrap 400 is secured to the limb of the patient, the straps are wrapped around the limb and the hooks on the first side of the strap 402 engage with loops on the second side of the straps 402.

In some embodiments, a first portion of a side of the strap 402 may have hooks and a second portion of the same side may have loops. In such an embodiment, the straps 402 pass through the aperture 405 in the ring 403 and then looped back such that the first portion of the strap 402 with the hooks is coupled to the second portion of the strap 402 having the loops.

FIG. 6 shows an embodiment of a multi-pocket wrap 500. The wrap 500 includes three pockets 512A, 512B, 512C. Each pocket 512 is formed between respective lower and upper material layers of the wrap 500, similar to how the single pocket 112 is formed between lower and upper material layers 106, 110 in the wrap 100 shown in FIGS. 1A, 1B, 1C. The separate pockets 512 allow a medical professional to provide different partial vacuum pressures and flow rates in the region proximate each respective pocket 512.

Each pocket 512 may be in fluid communication with a respective port 504 that may be connected to a vacuum source, such as the vacuum source 630, shown in FIG. 2. In addition, each of the pockets 512 include fenestrations 520 that couple the interior of the pocket 512 in fluid communication with the environment external to the wrap 601. For example, during use the fenestrations 512 may be placed over the wound such that the interior of the pocket 512 in fluid communication with the region proximate or over to the wound on the patient.

A warp made in accordance with the disclosure herein was used in a pilot trail with two patients that suffered from long term open wounds that did not respond to other available treatments. The patients had mixed etiology venous, arterial and neuropathic disease. The two patients had a total of six wounds. One wound a diabetic ulcer, was 30 cm² in area and was resolved in four weeks. Four of the six wounds in the study were reduced in size in after four weeks of treatments. The treatment was carried out 1 to 2 times per week for 30 minutes and consisted of securing the wrap to the patient at the wound location, connecting the wrap to a vacuum source, such as a portable suction machine and applying 80-125 mmHg negative pressure at the suction machine for 30 minutes.

After less than 12 weeks of treatment all 6 wounds have healed such that treatment is no longer indicated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A wrap for treating wounds of a patient, the wrap comprising:
a first material layer;
a second material layer;
a first pocket formed between the first material layer and the second material layer;
a dressing located within the first pocket, between the first material layer and the second material layer; and
a first fluid port extending through the first material layer to the first pocket.

2. The wrap of claim 1, wherein the first material layer is a non-permeable layer.

3. The wrap of claim 1, wherein the second material layer is a permeable layer.

4. The wrap of claim 1, wherein the first material layer is a non-permeable layer and the second material layer is a permeable layer.

5. The wrap of claim 1, further comprising:
fenestrations formed through the second layer, the fenestrations coupling the first pocket in fluid communication with an environment outside the pocket.

6. The wrap of claim 3, further comprising:
fenestrations formed through the second layer.

7. The wrap of claim 1, further comprising:
saline solution within the dressing.

8. The wrap of claim 1, further comprising:
a first tie extending from a first end of the wrap and a second tie extending from a second end of the wrap, the ties configured to knot together to secure the wrap to a limb of a patient.

9. The wrap of claim 1, further comprising:
a re-sealable closure configured to open to provide access to the dressing.

10. The wrap of claim 1, further comprising:
a second pocket, separate from the first pocket formed between the first material layer and the second material layer;
a second dressing located within the second pocket, between the first material layer and the second material layer; and
a second fluid port extending through the first material layer to the second pocket.

11. A system for treatment of wounds of a patient, the system comprising:
a vacuum source;
a wrap including:
a first pocket in fluid communication with an environment external to the first pocket;
a dressing located within the first pocket; and
a first fluid port coupling the first pocket in fluid communication with the vacuum source; and
a fluid separator between the vacuum source and the wrap and coupled in fluid communication with the vacuum source and the wrap.

12. The system of claim 1, wherein the fluid separator is a filter.

13. The system of claim 11, wherein the fluid separator is a fluid trap.

14. The system of claim 11, wherein a permeable material defines a portion of a boundary of the first pocket and couples the first pocket in fluid communication with the environment external to the pocket.

15. The system of claim 11, wherein fenestrations in a material layer that defines a portion of a boundary of the first pocket couples the first pocket in fluid communication with an environment external to the first pocket.

16. The system of claim 11, further comprising:
a second pocket, mechanically coupled to, but fluidically separated from the first pocket;
a second dressing located within the second pocket; and
a second fluid port coupling the second pocket in fluid communication with the vacuum source.

17. The system of claim 11, wherein a boundary defining the pocket is formed from a non-permeable.

18. The system of claim 11, wherein the wrap further comprises:
a re-sealable closure configured to open to provide access to the first pocket.

19. The system of claim 11, wherein the wrap includes a plurality of ties extending from first and second ends of the wrap.

20. The system of claim 11, further comprising a first tube connecting the wrap in fluid communication with the fluid separator and a second tube connecting the fluid separator with the vacuum source.
